(19) United States
(12) Patent Application Publication
(10) Pub. No.: US 2006/0057722 A1
(43) Pub. Date: Mar. 16, 2006

(54) Conditioned medium of autologous or allogenic progenitor cells for angiogenesis treatment

(75) Inventors: Ran Kornowski, Ramat-Hasharon (IL); Shmuel Fuchs, Rockville, MD (US); Stephen E. Epstein, Rockville, MD (US); Martin B. Leon, New York, NY (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: MYOCARDIAL THERAPEUTICS, INC., La Jolla, CA (US)

(21) Appl. No.: 11/221,469

(22) Filed: Sep. 7, 2005

Related U.S. Application Data
(63) Continuation-in-part of application No. 10/618,183, filed on Jul. 10, 2003.
Application No. 10/618,183 is a continuation-in-part of application No. 10/160,514, filed on May 30, 2002, now abandoned.
Application No. 10/160,514 is a continuation-in-part of application No. 09/868,411, filed on Jun. 14, 2001.
(60) Provisional application No. 60/608,272, filed on Sep. 8, 2004.
(60) Provisional application No. 60/138,379, filed on Jun. 9, 1999.
(60) Provisional application No. 60/126,800, filed on Mar. 30, 1999.

(51) Int. Cl. C12N 5/08

(52) U.S. Cl. 435/372; 514/44

(57) ABSTRACT

A therapeutic composition is provided that comprises a cell-free conditioned medium containing mixed secretion products of isolated angiogenic progenitor cells obtained from bone marrow, peripheral blood, or adipose tissue. The composition may additionally contain angiogenesis-promoting proteins obtained by transfecting the progenitors cells in culture with an angiogenesis promoting transgene. The composition is useful to promote angiogenesis when introduced into or adjacent to an ischemic site in a patient, such as in myocardium or peripheral limb. Methods are also provided for utilizing such cell-free conditioned medium to deliver angiogenesis-promoting proteins to a patient. The cell-free conditioned medium can also be injected into the blood stream for delivery to the ischemic tissue. The cells can derive from either an autologous or allogenic source and can be lyophilized or frozen for storage.

### RELATED APPLICATIONS

This application claims the benefit of priority under 35 U.S.C. § 119(e) of U.S. Ser. No. 60/608,272, filed Sep. 8, 2004; which is related to Continuation-in-Part Application of U.S. patent application Serial No, 10/618,183, filed Jul. 10, 2003, which is a Continuation-in-Part Application of U.S. patent application Ser. No. 10/160,514, filed May 30, 2002, which is a Continuation-in-Part Application of U.S. patent application Ser. No. 09/868,411, filed Jun. 14, 2001, which was the National Stage of International Application No. PCT/US00/08353, filed Mar. 30, 2000, which relies for priority upon U.S. Provisional Patent Application Ser. Nos. 60/138,379, filed Jun. 9, 1999, and 60/126,800, filed Mar. 30, 1999, each of which is incorporated herein by reference in its entirety.

### FIELD OF THE INVENTION

This application is directed generally to methods of using bone marrow cells in treatment of various diseases and more specifically to use of conditioned medium derived from autologous and allogenic angiogenic progenitor cells to enhance collateral blood vessel formation (angiogenesis) and tissue perfusion. Although this application speaks to use of bone marrow cells, it is also intended to apply to conditioned medium from angiogenic progenitor cells in general, including cells isolated from the peripheral blood or from other tissues, including adipose tissue.

### BACKGROUND OF THE INVENTION

The use of recombinant genes or growth factors to enhance myocardial collateral blood vessel function may represent a new approach to the treatment of cardiovascular disease. Komowski, R., et al., “Delivery strategies for therapeutic myocardial angiogenesis,” Circulation 2000; 101:454-458. Proof of concept has been demonstrated in animal models of myocardial ischemia, and clinical trials are underway. Unger, E. F., et al., “Basic fibroblast growth factor enhances myocardial collateral flow in a canine model,” Am J Physiol (1994) 266:H1588-1595; Banai, S. et al., “Angiogenic-induced enhancement of collateral blood flow to ischemic myocardium by vascular endothelial growth factor in dogs,” Circulation (1994) 83:2189; Lazarous, D. F., et al., “Effect of chronic systemic administration of basic fibroblast growth factor on collateral development in the canine heart,” Circulation (1995) 91:145-153; Lazarous, D. F., et al., “Comparative effects of basic development and the arterial response to injury,” Circulation (1996) 94:1074-1082; Giordano, F. J., et al., “Intracoronary gene transfer of fibroblast growth factor-5 increases blood flow and contractile function in an ischemic region of the heart,” Nature Med (1996) 2:534-9. Guzman, R. J., et al., “Efficient gene transfer into myocardium by direct injection of adenovirus vectors,” Circ Res (1993) 73:1202-7; Mack, C. A., et al., “Biologic bypass with the use of adenovirus-mediated gene transfer of the complementary deoxyribonucleic acid for VEGF-121, improves myocardial perfusion and function in the ischemic porcine heart,” J Thorac Cardiovasc Surg (1998) 115:168-77.

For example, the effect of direct intra-operative intramyocardial injection of angiogenic factors on collateral function has been studied in animal models of myocardial ischemia. Open chest, transepicardial administration of an adenoviral vector containing a transgene encoding an angiogenic peptide resulted in enhanced collateral function. (Mack et al., supra.) Angiogenesis was also reported to occur with direct intramyocardial injection of an angiogenic peptide or a plasmid vector during open-heart surgery in patients. Schumacher, B., et al., “Induction of neoangiogenesis in ischemic myocardium by human growth factors. First clinical results of a new treatment of coronary heart disease,” Circulation (1998) 97:645-650; Losordo, D. W., et al., “Gene therapy for myocardial angiogenesis: initial clinical results with direct myocardial injection of phVEGF165 as sole therapy for myocardial ischemia,” Circulation (1998) 98:2800.

Despite the promising hope for therapeutic angiogenesis as a new modality to treat patients with circulatory disease in the heart or peripheral limbs, there is still a need in the art for new and better therapeutic strategies that will optimally promote a clinically relevant therapeutic angiogenic response.

### SUMMARY OF THE INVENTION

The present invention is based on the premise that multiple complex processes, involving the differential expression of dozens if not hundreds of genes, are necessary for optimal collateral development. Based on this concept, it follows that optimal development of collateral blood vessels and tissue perfusion cannot be achieved by the administration of single proteins, or single genes whose encoded products are known to be related to angiogenesis nor, because of the complexity of the angiogenesis processes, by the administration of a combination of angiogenesis-related proteins or genes. This invention relies on the capacity of certain angiogenic progenitor cells to secrete into growth medium the growth factors and cytokines involved in angiogenesis and collateral blood vessel formation in a time and concentration-dependent coordinated and appropriate sequence.

Most currently tested therapeutic approaches have focused on a single angiogenic growth factor (e.g., VEGF, FGF, angiopoietin-1) delivered to the ischemic tissue. This can be accomplished either by delivery of the end product (e.g., protein) or by gene transfer, using diverse vectors. However, it is believed that complex interactions among several growth factor systems are probably necessary for the initiation and maintenance of new blood vessel formation. More specifically, it is believed important to induce a specific localized angiogenic milieu with various angiogenic cytokines interacting in concert and in a time-appropriate manner to initiate and maintain the formation and function of new blood vessels.

Accordingly, in one embodiment, the invention provides methods for producing a composition useful for enhancing development of collateral blood vessels in a patient in need by growing isolated autologous or allogenic angiogenic progenitor cells under suitable culture conditions in a suitable medium for a period of time sufficient to promote production by the angiogenic progenitor cells of conditioned medium containing mixed secretion products. The condition medium is processed to a cell-free conditioned medium comprising the mixture of mixed secretion products of the angiogenic progenitor cells. When injected into a site within or adjacent to tissue having impaired blood flow in a patient, a composition that includes the cell-free medium promotes development of collateral blood vessels in the tissue

In another embodiment, the invention provides a therapeutic composition useful for enhancing development of collateral blood vessels in a patient having a site of impaired blood flow when injected into to a site of developing collaterals that supply the tissue with impaired blood flow. The invention therapeutic composition includes a cell-free medium containing a mixture of cytokines, wherein the cell-free medium is produced by growing isolated allogenic donor progenitor cells in a suitable growth medium and under conditions suitable to promote production by the progenitor cells of the mixture of growth products. The conditioned medium is then processed to remove the cells therefrom to yield the cell-free conditioned medium.

In still another embodiment, the invention provides a kit that includes the invention therapeutic composition contained in a container; and an instruction for using the composition to enhance collateral blood vessel development within or adjacent to a site of impaired blood flow in a mammal.

In yet another embodiment, the invention provides methods for enhancing collateral blood vessel formation in a patient having a site of impaired blood flow by directly administering an amount of the invention composition sufficient to enhance collateral blood vessel formation to a site of developing collaterals that supply the tissue having impaired blood flow in the patient, such as ischemic tissue of heart or limb

### BRIEF DESCRIPTION OF THE DRAWINGS

FIG. 1 is a graph of the proliferation of pig aortic endothelial cells (PAECs) vs. the quantities of conditioned medium.

FIG. 2 is a graph of the proliferation of endothelial cells vs. the quantities of conditioned medium.

FIG. 3 is a graph of the concentration of VEGF in conditioned medium over a four-week period of time.

FIG. 4 is a graph of the concentration of MCP-1 in conditioned medium over a four-week period of time.

FIG. 5 is a graph showing in-vitro production of VEGF, MCP-1 and bFGF by CD34+ cells and bone marrow-derived stromal cells from mice.

FIG. 6 is a graph showing the effect of bone marrow-derived stromal cells on development of collateral flow when injected into adductor muscles of ischemic hind limb of mice as determined by Laser/Doppler perfusion imaging. Flow is expressed as the ratio of flow in the ischemic limb to flow in the normal hindlimb. MSC=marrow-derived stromal cell; Media=non-conditioned media; MAEC=mouse aortic endothelial cells.

FIG. 7 is a graph showing the effect on release of VEGF and bFGF in vitro from mouse marrow-derived stromal cells (MSCs) transfected with an adenovirus encoding HIF-1I-VP16. (MSC=MSCs alone; hypoxia=hypoxia conditions alone; HIF=MSCs transfected with DNA encoding fusion protein HIF-1I-VP16. Data represent analysis of at least 3 different MSC populations.

FIG. 8 is a graph showing improved in vivo flow recovery in mice receiving 1×10⁵ HIF-1I/VP16 transduced MSCs injected into an ischemic hind limb compared to 1×10⁵ non-transduced MSCs (comparison of trends p=0.05 by ANOVA). Cells injected on day 1.

### DETAILED DESCRIPTION OF THE INVENTION

The bone marrow (BM) is a natural source of a broad spectrum of cytokines (e.g., growth factors) and cells that are involved in the control of angiogenic processes. Delivery of autologous (A) BM or bone marrow cells derived therefrom, or media derived from these cells while the cells are grown in culture, by taking advantage of the natural ability of these cells to secrete many angiogenic factors in a time-appropriate manner, provides an optimal intervention for achieving therapeutic collateral development in ischemic myocardium and peripheral limb as well as in other tissue experiencing impaired blood flow.

However, the time required to obtain and culture autologous bone marrow may unduly delay the use of bone marrow for treatment of such conditions. In certain instances, the age of the patient may make use of autologous bone marrow unsatisfactory. In the case of myocardial infarction, time may be an important factor to consider. However, it is known that due to immune response, use of any but autologous cells requires finding a “matching” donor whose cells will not be rejected by the patient.

The present invention is based on the discovery that cell-free medium derived from growth in vitro of autologous or allogenic progenitor cells, such as, but not limited to, those obtained from bone marrow, can be used in the place of the cells themselves because this conditioned medium provides delivery to the tissue of a patient the many angiogenic factors secreted by progenitor cells that participate in growth of collateral vessels in tissue. Suitable progenitor cells for use in the invention methods and compositions may also be obtained, for example, from peripheral blood or from other tissues, including adipose tissue. The invention cell-free medium is produced by culturing isolated allogenic or autologous progenitor cells under suitable conditions and for a time sufficient for the progenitor cells to secrete mixed secretion products into the conditioned medium. The conditioned medium is then processed to yield a cell-free medium containing the mixed secretion products. As with preparation of donor blood for transfusion, in which only the red cells are typed and cross-matched, the other cells administered, as well as the plasma, are administered without any typing. The incidence of serious allergic responses to any of these products is very low. The cell-free conditioned medium derived from cells of an allogenic or autologous source can be considered as free of allergens as serum or plasma obtained from various donors.

The cytokines remaining in the cell-free medium are relatively small molecules as compared with the size of many proteins and, therefore, lack features that the mammalian body recognizes as non-self, leading to immune response. The size differential between cells and cytokines makes it convenient to remove the cells from the growth medium to yield the cell-free medium by filtering the growth medium or by centrifugation, for example for five minutes at 10 k×g. The cell-free medium may be further processed, such as by freezing or lyophilization, and placed into small containers to make handling, storage and distribution convenient. Those of skill in the art would understand that the frozen or lyophilized cell-free medium would be readily reconstituted for use by addition of such fluids as sterilized water, physiological saline, and the like, using the techniques know in the art as suitable for preparing other types of blood cells and blood products for administration to a patient.

The bone marrow (BM) is a natural source of a broad spectrum of cytokines (e.g., growth factors), various factors and cells that are involved in the control of angiogenic processes, which are referred to herein collectively as “mixed secretion products” for convenience. It is therefore believed that the intramyocardial injection of autologous (A) BM or bone marrow cells derived therefrom, by taking advantage of the natural ability of these cells to secrete many angiogenic factors in a time-appropriate manner, provides an optimal intervention for achieving therapeutic collateral development in ischemic myocardium.

The present invention represents an advance in the art by taking advantage of the discovery that autologous or allogenic progenitor cells associated with angiogenic development can be used to prepare cell-free conditioned medium containing endogenously secreted mixed secretion products. In addition, at least some of such autologous or allogenic progenitor cells can be transfected with polynucleotides encoding one or more of the angiogenic proteins (i.e., cytokines, growth factors, or transcription factors that enhance the capacity of target tissue to develop collateral blood supply to an area of ischemic tissue). Such cell transfection will boost the concentration of one or more of the mixed secretion products of the cells or add one or more additional angiogenic or arteriogenic proteins to the conditioned medium produced by the cells. During growth of the transfected progenitor cells in vitro, these gene products will be added to the conditioned medium and, thus, further contribute to the therapeutic effect of conditioned medium generated by growing the autologous or allogenic progenitor cells. Non-limiting examples of these mixed factors and growth products are Granulocyte-Monocyte Colony Stimulatory Factor (GM-CSF), Monocyte Chemoattractant Protein-1 (MCP-1), and Hypoxia Inducible Factor-1 (HIF-1). The transfected progenitor cells will be removed from the conditioned medium during processing to produce a cell-free conditioned medium.

Alternatively or additionally, such progenitor cells, obtained as described herein, can be transfected with a gene encoding one or species of nitric oxide synthase (NOS). A “NOS gene” or a “polynucleotide encoding NOS” as these terms are used herein mean a gene encoding any of the known isoforms of NOS, including inducing NOS (iNOS) and endothelial NOS (eNOS), as well as NOS genes that have been mutated such that the magnitude of their expression is altered, or so that they encode an altered protein, either of which results in a more potent angiogenic effect.

The rationale for transducing at least some of the progenitor cells with a polynucleotide encoding NOS is based on the fact that VEGF, one of the more potent angiogenic agents identified, works through NOS signaling pathways. For example, it has been shown that VEGF fails to induce angiogenesis in mice in which a NOS gene has been knocked out. Moreover, nitric oxide (NO), the protein product of NOS, has multiple actions that induce angiogenesis and, moreover, induce the expression of many different genes, many of which are involved in angiogenesis. Thus, transfecting progenitor cells with a polynucleotide encoding NOS augments the intrinsic capacity of the cells to secrete the mixed secretion products described herein and also stimulates expression of multiple angiogenesis-related genes.

Another family of genes this invention describes as having the capacity to augment the potential of progenitor cells to enhance collateral blood vessel development is the family of fibroblast growth factors (FGFs). This family of genes comprises over fourteen closely related genes including, but not limited to, FGF 1, FGF 2, FGF 4, and FGF 5. The rationale for transducing bone marrow cells with a gene in the FGF family is that FGF is known to be a potent stimulator of angiogenesis. FGF also stimulates the expression of multiple additional genes, many of whose protein products are capable of inducing angiogenesis.

The PR39 gene, expressed by monocytes/macrophages, is also suitable for transfer into progenitor cells, as described herein, to enhance the potential of cell-free conditioned medium produced thereby to improve collateral formation. The rationale for enriching the conditioned medium with the gene product of PR39 is that this protein inhibits proteasomal degradation of HIF-1α, resulting in accelerated formation of vascular structures in vitro and increased myocardial vasculature in mice. Increasing the steady state levels of HIF-1α, induces increased formation of the heterodimer—HIF-1α/HIF-1β, which is a transcription factor that induces the expression of HIF-1-related genes. The protein products of many of these genes promote the development of angiogenesis. The rationale for this strategy—increasing the steady-state levels of HIF-1α—has been described in detail above.

The progenitor cells used in preparation of the invention conditioned medium can be transfected, ex vivo, with a plasmid vector, or with an adenoviral vector, carrying an angiogenic cytokine growth factor or mammalian angiogenesis promoting factor transgene, such as the HIF-1 or EPAS1 transgene, or a transgene encoding PR39, or a member of the NOS or FGF families, for expression thereof into the conditioned medium derived from growing the cells. The medium so derived is processed to obtain cell-free conditioned medium and injected into a treatment site to improve angiogenesis as described herein.

Inoculation of the cells takes place after culture of the cells for a period of several hours in the presence of one or more vectors containing one or more transgenes, and the inoculated cells begin to produce the transgene products after about 12 hours to 3 days. Alternatively, the progenitor cells can be inoculated with a vector encoding one or more angiogenic cytokines, growth factors and/or factors that promote angiogenesis in mammalian cells by any method known in the art. The vectors used can be selected from any of those known in the art and include, but without limitation thereto, those described herein. Suitable culture conditions are well known in the art and include, but are not limited to, those described in the Examples herein.

An effective amount of the cell-free conditioned medium obtained by growth of transfected or untransfected progenitor cells as described herein can be directly administered to (i.e. injected into) a desired site in a patient to enhance collateral blood vessel formation at the site in the patient. Particularly effective sites for administration of the invention conditioned medium include heart muscle or skeletal muscle, such as in the leg, to enhance collateral-dependent perfusion in cardiac and/or peripheral ischemic tissue. The cell-free conditioned medium derived from such cells can also be injected into the vascular system so that the mixed secretion products and optional stimulatory angiogenic proteins contained therein are delivered to the desired site by the blood.

The polynucleotide encoding the stimulatory angiogenic protein may be “functionally appended to”, or “operatively associated with”, a signal sequence that can “transport” the encoded product across the cell membrane. A variety of such signal sequences are known and can be used by those skilled in the art without undue experimentation.

Gene transfer vectors (also referred to as “expression vectors”) contemplated for such purposes are recombinant nucleic acid molecules that are used to transport nucleic acid into host cells for expression and/or replication thereof. Expression vectors may be either circular or linear, and are capable of incorporating a variety of nucleic acid constructs therein. Expression vectors typically come in the form of a plasmid that, upon introduction into an appropriate host cell, results in expression of the inserted nucleic acid.

Suitable viral vectors for use in gene therapy have been developed for use in particular host systems, particularly mammalian systems, and include, for example, retroviral vectors, other lentivirus vectors such as those based on the human immunodeficiency virus (HIV), adenovirus vectors, adeno-associated virus vectors, herpesvirus vectors, vaccinia virus vectors, and the like (see Miller and Rosman, BioTechniques 7:980-990, 1992; Anderson et al., Nature 392:25-30 Suppl., 1998; Verma and Somia, Nature 389:239-242, 1997; Wilson, New Engl. J. Med. 334:1185-1187 (1996), each of which is incorporated herein by reference). Preferred gene transfer vectors are replication-deficient adenovirus carrying one or more of the transgenes that effect development of collateral arteries in a subject, which have been used successfully in subjects suffering progressive arterial occlusion (Barr et al., “PCGT Catheter-Based Gene Transfer Into the Heart Using Replication-Deficient Recombinant Adenoviruses,” Journal of Cellular Biochemistry, Supplement 17D, p. 195, Abstract P101 (March 1993); Barr et al., “Efficient catheter-mediated gene transfer into the heart using replication-defective adenovirus,” Gene Therapy (1994) 1:51-58).

Several different gene transfer approaches are feasible, including the helper-independent replication deficient human adenovirus 5 system. The recombinant adenoviral vectors based on the human adenovirus 5 (Virology (1988) 163:614-617) are missing essential early genes from the adenoviral genome (usually E1A/E1B), and are therefore unable to replicate unless grown in permissive cell lines that provide the missing gene products in trans. In place of the missing adenoviral genomic sequences, a transgene of interest can be cloned and expressed in tissue/cells infected with the replication deficient adenovirus. Although adenovirus-based gene transfer does not result in integration of the transgene into the host genome (less than 0.1% adenovirus-mediated transfections result in transgene incorporation into host DNA), and therefore is not stable, adenoviral vectors can be propagated in high titer and transfect non-replicating cells well. Studies have shown that only transient expression of the angiogenesis-promoting transgene is required to effect enhanced collateral development in ischemic heart or skeletal muscle into which the adenoviral vector is administered.

However, it is a particular feature of the present invention that the vector and transgene of interest will be removed from the conditioned medium during processing to obtain a cell-free conditioned medium, thus substantially avoiding any problems that might be encountered in gene therapy applications.

The amount of exogenous nucleic acid introduced into at least some of the angiogenic progenitor cells can be varied by those of skill in the art according to known principles. For example, when a viral vector is employed to achieve gene transfer, the amount of nucleic acid introduced to the cells to be transfected can be varied by varying the amount of plaque forming units (PFU) of the viral vector.

The cell-free conditioned medium of autologous or allogenic progenitor cells, alone or with stimulatory angiogenic proteins, can be delivered to the patient directly via either trans-endocardial or trans-epicardial approaches into either ischemic and/or non-ischemic myocardium, or directly into any other ischemic tissue (including a peripheral limb) to enhance and/or promote the development of collateral blood vessel formation and, therefore, collateral flow to ischemic myocardium or ischemic limbs. This approach can also be used to promote the development of newly implanted dedifferentiated and/or differentiated myocardial cells by the process of cardiac myogenesis.

Thus, according to various embodiments of the invention, the cell-free conditioned medium derived from growing progenitor cells in culture, whether the cells have been transfected or not with a transgene to further enhance their production of angiogenic proteins, is injected, either as a “stand alone” therapeutic composition or combined with any suitable pharmacologic drug or additional cytokine. For example, the cell-free medium may be supplemented by addition of an angiogenic growth factor that promotes development and formation of blood vessels. For example, it has been discovered that after growth in vitro for 7 to 10 (or more) days, bone marrow-derived progenitor cells (there may be only several cell lines growing after this period of time) secrete numerous cytokines-an effect that can be augmented when the cells are exposed, in vitro, to hypoxia or contacted with HIF-1, especially HIF-1α, or MCP-1, or other molecules that stimulate cell-signaling pathways involved in cellular response to hypoxia. When injected into tissue adjacent to an area of impaired blood flow, or tissue containing developing collaterals supplying such ischemic tissue, these secreted cytokines in the conditioned medium then stimulate the growth and remodeling of blood vessels. These may be new vessels (angiogenesis) or vessels that are present in the tissue, but too small to result in substantial flow (arteriogenesis). Thus, the invention does not rely on transdifferentiation of the cells of the tissue into which the invention therapeutic composition is injected, but in stimulating the formation of new blood vessels or expansion of existing but very small blood vessels. This concept has been tested in the laboratory, and shown to be valid.

As used herein, the term “bone marrow cells” means any cells that are produced by growth of aspirated bone marrow under cell growth conditions. Surprisingly, after 7-10 (or more) days of growth in suitable growth medium, as described in the Examples herein and as known in the art, the existing cell lines dwindle to a few progenitor cell lines. These bone marrow-derived progenitor cell line(s) are responsible for secreting the mixed secretion products into conditioned medium and can be isolated as described in the examples herein, and as known in the art. The conditioned medium can be harvested after 7-10 days of growth, or the existing medium can be removed and discarded, and the cells can be cultured for an additional 1-7 (or more) days and the new conditioned medium then can be harvested, processed to produce a cell-free medium and used as the invention.

Optionally, the bone marrow progenitor cells can be isolated from an early cell growth medium using cell screening techniques based on the presence of at least one identifying surface marker. For example, angiogenic progenitor cells obtained from bone marrow can be isolated from an initial growth medium, for example, by sorting out the CD34⁺ cells. Alternatively, the CD34⁻ cells can be sorting for and selected. The isolated cells are then grown to produce the conditioned medium as described herein. Similar (but not necessarily identical) methods can be used if the progenitor cells are derived from other tissues, including peripheral blood and adipose tissue. The isolated cells are then grown to produce the conditioned medium as described herein.

Non-limiting examples of the mixture of cytokines secreted by progenitor cells during growth in culture are VEGF, FGF, Monocyte Chemoattractant Protein (MCP-1), Macrophage-specific Colony Stimulating Factor (M-CSF) and placenta-derived growth factor (PlGF). A more complete table of secreted cytokines is found in Table 1 below.

TABLE 1
Expression of Proangiogenic/Proarteriogenic Gene
Products by Marrow-Derived Stromal Cells

| | Angiogenic/Arteriogenic | Fold Induction |
|---|---|---|
| Cytokine | Function | With Hypoxia |
| Angiopoietin-1 | EC migration, vessel | — |
| | stabilization | |
| Fibroblast growth | EC and SMC proliferation | 1.62 |
| factor-2 | and migration | |
| Fibroblast growth | EC proliferation and | 1.82 |
| factor-7 | stabilization | |
| Hepatoma growth factor | SMC proliferation | — |
| Interleukin-1 | VEGF induction | 1.91 |
| Interleukin-6 | VEGF induction | 2.26 |
| Metalloproteinase-1 | Loosens matrix, tubule | — |
| | formation | |
| Metalloproteinase-2 | Loosens matrix, tubule | — |
| | formation | |
| Metalloproteinase-9 | Loosens matrix | — |
| MCP-1 | Monocyte migration | — |
| M-CSF | Monocyte proliferation/ | — |
| | migration | |
| Placental growth | EC proliferation | 2.93 |
| factor | | |
| Plasminogen activator | Degrading matrix | — |
| | molecules | |
| Platelet-derived | SMC proliferation and | — |
| growth factor | migration | |
| Stem cell-derived | Progenitor cell homing | — |
| factor | | |
| Transforming growth | Vessel maturation, EC | 2.11 |
| factor-θ | proliferation | |
| Tumor necrosis | Degrade matrix molecules, | 1.69 |
| factor-I | EC proliferation | |
| VEGF-A | EC proliferation, migration, | 2.47 |
| | tube formation | |
| VEGF-B | EC proliferation, migration, | — |
| | tube formation | |

MCP-1 = monocyte chemoattractant protein-1; M-CSF = macrophage-specific colony-stimulating factor; VEGF = vascular endothelial growth factor; EC = endothelial cell; and SMC = smooth muscle cell.

Research indicates that angiogenesis is needed to support bone marrow function and development of hematopoietic cells, including stem cells and progenitor cells, which may enter the circulation and target to sites of wound healing and/or ischemia, ultimately contributing to new blood vessel formation. Monoclonal antibodies that specifically recognize undifferentiated mesenchymal progenitor cells isolated from adult human bone marrow have been shown to recognize cell surface markers of developing microvasculature, and evidence suggests such cells may play a role in embryonal angiogenesis (Fleming, J. E., Jr., Dev Dyn (1998) 212:119-32).

Thus, it is believed that progenitor cells obtained from donor bone marrow, adipose tissue or peripheral blood, or a combination thereof, provide a natural source of “mixed secretion products”, and that conditioned medium produced by growing such isolated progenitor cells can surprisingly be utilized to produce therapeutic angiogenesis due to the presence of a mixture of potent interactive growth factors therein. In addition, it has now been surprisingly discovered that the cell-free conditioned media in which such isolated progenitor cells are cultured contains mixed secretion products, including growth factor proteins, which produce therapeutic angiogenesis and/or myogenesis. Moreover, therapeutic effects for a patient suffering impaired blood flow, such as in ischemic tissue of heart or peripheral limb, can be produced by administering to the affected tissue, or to adjacent unaffected tissue, cell-free conditioned medium produced by culturing such isolated autologous or allogenic progenitor cells for a time suitable to allow secretion into the growth medium by the progenitor cells of mixed secretion products, and processing the growth medium to remove the cells to produce therapeutic angiogenesis and/or myogenesis resulting in development of collateral blood vessels.

It has been previously demonstrated that stromal cells derived from human subcutaneous adipose tissue will support hematopoiesis (Storms et al. Blood (2000) 96:685a, and Blood (2001) 98:85 1a). It is also known that circulating progenitor cells can be collected from peripheral blood. Numbers of circulating progenitor cells (PBPCs) can be significantly increased in these protocols by pre-treatment of the donor with hematopoietic growth factors. Following such mobilization, only one to three aphaereses are needed to obtain sufficient cells from a donor for culturing. For use in the invention methods, the PBPCs are collected by standard aphaeresis techniques, or other standard techniques, and cryopreserved in liquid nitrogen. Compared to bone marrow harvest, autologous or allogenic PBPC collection can be done in the outpatient setting, requires no anesthesia, and can be repeated as often as needed to obtain sufficient progenitor cells for culturing to obtain cell-free conditioned medium for use in the invention treatment methods. Also, compared to bone marrow harvest, collection of autologous or allogeneic progenitor cells from adipose tissue can be done more simply.

DMSO is a cryoprotector usually used in protocols for freezing peripheral blood progenitor cells (PBPCs). If PBPCs are frozen prior to culturing, because DMSO can cause some undesired side effects when injected into patients, the thawing protocol consists of centrifuging thawed PBPCs for 1.5 min at 1250 g, and washing them once again at the same conditions, in order to wash-out DMSO. The wash solution is NaCl-glucose buffer+10% ACD. Total nucleated cell numbers and proportion of a specific cell type can be determined by standard FACS techniques.

Less well known as a source of progenitor cells is human adipose tissue (AT), which undergoes neovascularization associated with development of fat mass. In the human AT-derived stroma vascular fraction, the presence of a CD34⁺/CD31⁻ cell population has been identified and shown to exhibit in vitro the differentiation plasticity of stem cells. To demonstrate the angiogenic potential of such circulating progenitor cells (CPCs), a murine model of hindlimb ischemia has been used (A. Miranville et al., Institut fur Kardiovaskulare Physiologie, JW Goethe Universitat, Frankfurt am Main). Twenty four hours after ligation of the superficial and deep femoral artery of nude mice, freshly isolated CD34⁺/CD31⁻ cells were intravenously injected. After 2 weeks, a statistically significant increase in the relative blood flow of the ischemic limb was observed after injection of the CD34⁺/CD31⁻ cells as compared to injection of CD34⁻/CD31⁻ cells or injection of no cells. This study showed that AT-derived isolated CPCs from cross species can exert angiogenic effects. Another study has demonstrated that adipose stromal cells (ASCs), isolated from human subcutaneous adipose tissue, secreted 1203±254 pg of vascular endothelial growth factor (VEGF) per 10⁶ cells, 12 280±2944 pg of hepatocyte growth factor per 10⁶ cells, and 1247±346 pg of transforming growth factor-beta per 10⁶ cells. When ASCs were cultured in hypoxic conditions, VEGF secretion increased 5-fold (P=0.0016). Conditioned media obtained from hypoxic ASCs significantly increased endothelial cell growth (P<0.001). Nude mice with ischemic hind limbs demonstrated marked perfusion improvement (P<0.05) when treated with human ASCs. (Rehman J, et al. Circulation (2004) March 16;109(10): 1292-8). It is an aspect of this invention that the cell-free conditioned medium derived from such non-autologous cells can also be used to enhance collateral development of vessels in mammalian tissue.

One angiogenesis-promoting factor that most likely participates in initiating angiogenesis in response to ischemia is HIF-1, a potent transcription factor that binds to and stimulates the promoter of several genes involved in responses to hypoxia. Induction and activation of HIF-I is tightly controlled by tissue pO₂. HIF-1 expression increases exponentially as pO₂ decreases, thereby providing a positive feedback loop by which a decrease in pO₂ causes an increase in expression of gene products that serve as an adaptive response to a low oxygen environment. Activation of HIF-1 leads, for example, to the induction of erythropoietin, genes involved in glycolysis, and to the expression of VEGF. HIF-1 is thought to also modulate the expression of many other genes that participate in the adaptive response to low pO₂ levels. HIF-1 regulates levels of proteins involved in the response to hypoxia by transcriptional regulation of genes responding to low pO₂, which genes have short DNA sequences within the promoter or enhancer regions that contain HIF-1 binding sites, designated as hypoxia responsive elements (HRE).

It is relevant that while expression of HIF-1 (as determined in HeLa cells) is exponentially and inversely related to pO₂, the inflection point of the curve occurs at an oxygen saturation of 5%, with maximal activity at 0.5% and ½ maximal activity at 1.5-2.0%. Such relatively low levels of hypoxia may not occur in the presence of mild levels of myocardial or lower limb ischemia—i.e., levels present in the absence of tissue necrosis, such as myocardial infarction, and leg ulcerations, respectively) to upregulate expression of hypoxia inducible angiogenic genes to cause secretion by bone marrow cells of angiogenic factors and enhanced collateral development in such low hypoxia tissue environments. Therefore, in one embodiment the invention provides methods for stimulating production of hypoxia response genes in bone marrow cells by co administering 1) invention autologous or allogenic conditioned medium described herein, and 2) progenitor cells transfected with a gene encoding a modified form of HIF-1 that is not degraded in the presence of higher pO₂ levels and therefore is constitutively active.

Because of the lability of HIF-1α in the absence of hypoxia, to assure its constitutive activity even under normoxic conditions, a chimeric construct of the HIF-1α gene has been constructed, consisting of the DNA-binding and dimerization domains from HIF-1α and the transactivation domain from herpes simplex virus VP16 protein as described in Example 8 below. The VP16 domain abolishes the ubiquitination site in HIF-1I, and therefore eliminates the proteasomal-mediated degradation of the protein. Thus, the resulting stable levels of HIF-1α lead to constitutive transactivation of the genes targeted by HIF-1. Expression of this or functionally related forms of HIF (such as HIF-2), or of factors that effect the HIF pathway, resulting in functionally similar effects, will provide optimal expression of many of the hypoxia-inducible angiogenic genes present in the bone marrow derived conditioned medium. In yet other embodiments, the supplementary HIF-1 or related material can be added to progenitor cell-derived conditioned medium prior to its administration or HIF-1 can be separately injected, either as the protein, or as the gene. If as the latter, HIF-1 can be injected either in a plasmid or viral vector, or in any other manner that leads to the presence of functionally relevant protein levels.

The transcriptional activity of HIF-1 (or the HIF-1I construct) derives binding to a specific DNA hypoxia-responsive recognition element (HRE) present in the promoter of many genes involved in the response of the cell to hypoxia, including VEGF, VEGFR1, VEGFR2, Ang-2, Tie-1, and nitric oxide synthase. Thus, HIF-1 plays a pivotal role in coordinating the tissue response to ischemia.

It is emphasized, however, that HIF-1 is used as an example of an intervention that could enhance production of angiogenic substances by progenitor cells. This invention also covers use of other angiogenic agents, which by enhancing HIF-1 activity (i.e., prolonging its half-life), or by producing effects analogous to HIF-1, stimulate progenitor cells, for example those obtained from bone marrow, to increase expression of angiogenic factors.

In yet further embodiments, the invention therapeutic cell-free medium is prepared by exposure of isolated angiogenic progenitor cells to endothelial PAS domain protein 1 (EPAS1). EPAS1 shares high structural and functional homology with HIF-1 and is also known as HIF-2. Like HIF-1, supplementary EPAS1 can be directly added to progenitor cell-derived conditioned medium ex-vivo to stimulate angiogenic activity of the medium prior to injection of the medium or EPAS1 can be separately injected into a subject being treated according to the invention methods, either as the protein, or as the gene.

In another embodiment according to the invention, to stimulate VEGF promoter activity by HIF-1, autologous or allogenic progenitor cells can be exposed ex-vivo in growth medium to hypoxia or other forms of energy, such as, for example, ultrasound, RF, or electromagnetic energy. This intervention increases expression of VEGF and other genes.

Current data also indicate the importance of monocyte-derived cytokines for enhancing collateral function. Monocytes are activated during collateral growth in vivo, and monocyte chemotactic protein-1 (MCP-1) is upregulated by shear stress in vitro. It has been shown that monocytes adhere to the vascular wall during collateral vessel growth (arteriogenesis) and capillary sprouting (angiogenesis). MCP-1 was also shown to enhance collateral growth after femoral artery occlusion in the rabbit chronic hindlimb ischemia model (Ito et al., Circ Res (1997) 80:829-3). Activation of monocytes seems to play an important role in collateral growth as well as in capillary sprouting. Increased monocyte recruitment by LPS is associated with increased capillary density as well as enhanced collateral and peripheral conductance at 7 days after experimental arterial occlusion (M. Arms et al., J Clin Invest (1998) 101:40-50.).

Accordingly, a further aspect of the invention involves the ex-vivo stimulation by MCP-1, of autologous or allogenic progenitor cells during growth in a suitable medium as described herein followed by the direct delivery of acellular culture medium containing a mixture of cytokines secreted by the cells to the ischemic myocardium or peripheral organ or skeletal muscle (e.g., ischemic limb) to enhance collateral-dependent perfusion and muscular function in cardiac and/or peripheral ischemic tissue. The stimulation of the angiogenic progenitor cells can be by the direct exposure of the cells during growth to MCP-1 in the form of the protein.

Granulocyte-macrophage colony-stimulating factor (GM-CSF) and Granulocyte-Colony Stimulatory Factor (G-CSF) are stimulatory cytokines for monocyte maturation and are multipotent hematopoietic growth factors, which are utilized in clinical practice for various hematological pathologies, such as depressed white blood cell count (i.e., leukopenia or granulocytopenia or monocytopenia), which occurs usually in response to immunosuppressive or chemotherapy treatment in cancer patients. GM-CSF has also been described as a multilineage growth factor that induces in vitro colony formation from erythroid burst-forming units, eosinophil colony-forming units (CSF), and multipotential (CSF), as well as from granulocyte-macrophage CSF and granulocyte CFU. (Bot F. J., Exp Hemato (1989) 17:292-5). Ex-vivo exposure to GM-CSF has been shown to induce rapid proliferation of CD-34⁺ progenitor cells (Egeland T. et al., Blood (1991) 78:3192-g). These cells have the potential to differentiate into vascular endothelial cells and may naturally be involved in postnatal angiogenesis. In addition, GM-CSF carries multiple stimulatory effects on macrophage/monocyte proliferation, differentiation, motility and survival (reduced apoptotic rate). Consistent with the combined known effects on bone marrow derived endothelial progenitor cells and monocytes, it is another aspect of the invention to use GM-CSF as an adjunctive treatment to injections of cell-free conditioned medium derived by growing angiogenic progenitor cells in medium as described herein to induce new blood vessel formation and differentiation in ischemic cardiovascular organs. Moreover, GM-CSF may further enhance therapeutic myocardial angiogenesis caused by injection of the invention acellular conditioned medium, by augmenting the effect, as described herein, of agents such as HIF-1, EPAS 1, hypoxia, or MCP-1.

Thus, in one embodiment, the invention involves the ex-vivo stimulation of autologous or allogenic progenitor cells during growth or stimulation of cell-free conditioned medium produced by growth of such cells by at least one compound selected from HIF-1, EPAS1, MCP-1, GM-CSF or by direct exposure of autologous or allogenic progenitor cells used in production of the invention composition to hypoxic environment while the cells grow in culture. The cell-free conditioned medium produced by growth of the autologous or allogenic progenitor cells is delivered to the ischemic myocardium or peripheral organ or skeletal muscle (e.g., ischemic limb) to enhance collateral-dependent perfusion in cardiac and/or peripheral ischemic tissue.

However, autologous bone marrow cells that are injected into regions in which collateral blood vessel development is desired in order to enhance the delivery of blood to ischemic regions may not produce optimal angiogenic effects when certain “at risk” conditions prevail. For example, there is evidence demonstrating that angiogenesis is impaired in the presence of hypercholesterolemia, and it is also compromised with aging. In addition, there are a number of genetic and other disorders that impair naturally occurring angiogenic processes, including the function of bone marrow cells, as compared with that found in normal young healthy individuals.

Hypercholesterolemia is a dominantly inherited genetic condition that results in markedly elevated low-density lipoprotein cholesterol levels beginning at birth, and resulting in myocardial infarctions at an early age. “Aging” as the term is used herein is not necessarily measured in years, but is measured in terms of deterioration of the body's ability to maintain the vascular system in a healthy condition. Nevertheless, the ability of the body to maintain vascular health tends to deteriorate with time (i.e., with age) as well.

Experimental evidence suggests collateral development of the vasculature is impaired in the elderly, who represent the largest cohort of patients affected by advanced arteriosclerosis and tissue ischemia. For example, the functions of both bone marrow progenitor cells (BMPCs) and HIF-1 are reduced with aging. Therefore, all of the age-related factors that impair collateral development in elderly patients would also impair the activity of autologous angiogenic progenitor cells, such as bone marrow-derived cells, that are retrieved from older patients for processing and delivery to their ischemic tissue or for preparation of cell-free conditioned medium according to the invention.

These drawbacks can be overcome, according to the invention methods, by administering to such patients cell-free conditioned medium prepared by culturing isolated allogenic progenitor cells (derived from bone marrow cells, peripheral blood or adipose tissue) obtained from young healthy individuals, and processing the conditioned medium to remove cells therefrom to yield a cell-free conditioned medium.

When prepared from either autologous or allogenic bone marrow, the bone marrow can optionally be filtered prior to placement in the growth medium to remove particles larger than about 300μ to about 200μ. Bone marrow cells can also be separated from the filtered ABM for growth leading to production of progenitor cells. Usually the growth time required to move from bone marrow to a composition comprising only a few cell lines among which are one or a few progenitor cells lines is about 7 to 10 days. The bone marrow-derived progenitor cells can then be isolated, and additionally grown in a suitable growth medium for a suitable period of time, for example, about 24 hours, to secrete the mixed secretion products that enhance angiogenesis and development of collateral perfusion in ischemic tissue. The conditioned medium containing the mixed secretion products can be collected through a filter selected to remove cells or otherwise processed as is known in the art to substantially remove cells to produce a cell-free medium. Suitable culture conditions for both cell growth steps are illustrated, but are not limited to, those described in the Examples herein. Similar (but not necessarily identical) methods can be used if the progenitor cells are derived from other tissues, including peripheral blood and adipose tissue.

An “effective amount” of the cell-free medium containing angiogenic progenitor cell-secreted mixed secretion products, as the term is used herein, means an amount sufficient to stimulate development of collateral blood flow in an area experiencing reduced blood flow or ischemic conditions. The cell-free medium can be directly administered to (i.e. injected into) an ischemic site or into an area adjacent to an ischemic site in a patient to enhance collateral blood vessel formation at the site in the patient. Particularly effective sites for administration of the invention cell-free medium include heart muscle or skeletal muscle, such as in the leg, to enhance collateral-dependent perfusion in cardiac and/or peripheral ischemic tissue. The invention cell-free conditioned medium can also be injected into the vascular system for delivery to the desired site by the blood.

The phrase “marrow-derived stromal cells” as used herein means CD34 minus/CD45 minus that can be obtained from growing a sample of bone marrow. Similar, but not necessarily identical, cells can be obtained from tissues other than bone marrow.

The cell-free medium produced according to the invention may be delivered alone or in combination with additional angiogenic cytokines. The cell-free medium can be delivered to the patient directly via either trans-endocardial or trans-epicardial approaches (e.g., via a catheter) into either ischemic and/or non-ischemic myocardium, or directly into any other ischemic tissue (including a peripheral limb) to enhance and/or promote the development of collateral blood vessel formation and therefore collateral flow to ischemic myocardium or ischemic limbs. This approach can also be used to promote and/or support the development of new myocardium (cardiac myogenesis) through implantation of dedifferentiated and/or differentiated myocardial cells by enhancing the development of collaterals that would provide nutrient flow to the newly developed cardiac myocytes.

The invention comprises various strategies to enhance angiogenesis and thereby accelerate the development of new blood vessels into ischemic myocardium or lower extremities. Another aspect of the invention concerns the strategy of “optimization of angiogenic gene expression” by progenitor cells in vitro to produce conditioned medium. This strategy includes co-administration of, or transfection of the progenitor cells in vitro with, an oligonucleotide encoding an HIF-1 transcription factor to induce expression of multiple genes involved in the response to hypoxia. A similar approach involves co-administration with the invention acellular medium, or the transfection of autologous or allogenic progenitor cells in vitro, with a polynucleotide encoding endothelial PAS domain protein 1 (EPAS 1). The strategy also involves the ex-vivo exposure of the autologous or allogenic progenitor cells to hypoxia to increase the production of vascular endothelial growth factor (VEGF) and other angiogenic proteins whose expression is increased by hypoxia, or co-administration of the acellular conditioned medium with other cytokines having proven angiogenic activity (such as MCP-1) by direct injection into the heart or any peripheral ischemic tissue. This invention thus includes the direct intramyocardial (trans-epicardial or trans-endocardial) or peripheral intramuscular injection of acellular conditioned medium produced by growth of autologous or allogenic progenitor cells, stimulated autologous or allogenic progenitor cells, for example, stimulated by HIF-1, EPAS1, MCP-1, GM-CSF, or transient exposure to hypoxia or other forms of energy, such as ultrasound, RF, electromagnetic or laser energy. In certain embodiments of the invention, stimulation of the progenitor cells can be by the direct exposure of the autologous or allogenic progenitor cells to angiogenic factors in the form of proteins, such as any of the FGFs or VEGF.

The discovery that isolated autologous or allogenic progenitor cells obtained from bone marrow, adipose tissue or peripheral blood can be substituted for autologous bone marrow cells to produce conditioned medium with angiogenic-producing effects has led to the present invention. Moreover, the advantages of using allogenic donor-provided progenitor cells to produce the therapeutic cell-free conditioned medium are several. First, an ischemic or older patient does not have to undergo anesthesia to obtain autologous bone marrow. Bone marrow from a young healthy donor produces more vigorous progenitor cells and the processing of allogenic progenitor cells conditioned medium to remove cells renders the invention composition no more immunogenic than blood plasma. In addition, the therapeutic composition produced from allogenic progenitor cells can be produced in advance and stored for immediate use by a recipient patient. For example, the therapeutic composition can also be frozen or lyophilized to accommodate storage.

In the examples below, certain aspects of the invention are illustrated. These examples are intended to illustrate, but not to limit the invention.

### EXAMPLES

### Example 1

Effect of Bone Marrow Cultured Media on Endothelial Cell Proliferation

Studies were conducted to determine whether aspirated pig autologous bone marrow cells secreted VEGF, a potent angiogenic factor, and MCP-1, which recently has been identified as an important angiogenic co-factor. Bone marrow was cultured in vitro for four weeks. The conditioned medium produced by growth of the cells was added to cultured pig aortic endothelial cells (PAECs), and after four days proliferation was assessed. VEGF and MCP-1 levels in the conditioned medium were assayed using ELISA. During the four weeks in culture, BM cells secreted VEGF and MCP-1, such that their concentrations increased in a time-related manner. The resulting conditioned medium enhanced, in a dose-related manner, the proliferation of PAECs. The results indicate that BM cells are capable of secreting potent angiogenic cytokines, such as VEGF and MCP-1, and of inducing proliferation of vascular endothelial cells.

Pig Bone Marrow Culture

Bone marrow (BM) cells were harvested under sterile conditions from pigs with chronic myocardial ischemia in preservative free heparin (20 units/ml BM cells) and filtered sequentially using 300μ and 200μ stainless steel mesh filters. BM cells were then isolated by Ficoll-Hypaque gradient centrifugation and cultured in long-term culture medium (LTCM) (Stem Cell Tech, Vancouver, British Columbia, Canada) at 330° C. with 5% CO₂ in T-25 culture flask. The seeding density of the BMCs in each culture was 7×10⁶/ml. Weekly, one half of the medium was removed and replaced with fresh LTCM. The removed medium was filtered (0.2μ filter) and stored at −200° C. for subsequent Enzyme-linked Immunosorbent Assay (ELISA) and cell proliferation assays.

Isolation and Culture of Pig Aortic Endothelial Cells

Fresh pig aortic endothelial cells (PAECs) were isolated using conventional methods. Endothelial cell growth medium (EGM-2 medium, Clonetics, San Diego, Calif.), containing 2% FBS, hydrocortisone, human FGF, VEGF, human EGF, IGF, heparin and antibiotics, at 37° C. with 5% carbon dioxide was used for growth of the cells. When the cells became confluent at about 7 days, they were split by 2.5% trypsin and cultured thereafter in medium 199 with 10% FBS. Their identity was confirmed by typical endothelial cell morphology and by immunohistochemistry staining for factor VIII. Passage 3-10 was used for the proliferation study.

Effects of Conditioned Medium on Aortic Endothelial Cells

Cell proliferation assay: PAECs (Passage 3-10) were removed from culture flasks by trypsinization. The detached cells were transferred to 96-well culture plates and plated at a seeding density of 5,000 cells/well. Cells were cultured for 2-3 days before being used in proliferation and DNA synthesis experiments. The conditioned media of BM cell cultures were collected at 4 weeks; medium from 7 culture flasks were pooled and used in the bioassay. Aliquotes (10 μL, 30 μL, 100 μL or 200 μL) of pooled conditioned medium, or LTCM (200 μL, as control), were added to confluent PAECs in 96-well plates in triplicate. Four days following culture with conditioned medium or control medium, the PAECs were trypsinized and counted using a cell counter (Coulter Counter Beckman Corporation, Miami Fla.).

Effects of Conditioned Medium on PAEC DNA Synthesis

Aliquotes (10 μL, 30 μL, 100 μL or 200 μL) of conditioned medium from pooled samples or control medium (LTCM, 200 μL) were added to PAECs in a 96-well plate (same seeding density as above) in triplicate. After 2 days, 1 μCi tritiated thymidine was added to each well. Forty-eight hours later, DNA in PAECs was harvested using a cell harvester (Mach III M Tomtec, Hamden, Conn.) and radioactivity was counted by liquid scintillation counter (Multi-detector Liquid Scintillation Luminescence Counter EG&G Wallac, Turku, Finland).

Determination of VEGF and MCP-1 in Conditioned Medium by ELISA VEGF

The concentration of VEGF in conditioned medium was measured using a sandwich ELISA kit (Chemicon International Inc., Temecula, Calif.). Briefly, a plate pre-coated with anti-human VEGF antibody was used to bind to VEGF in the conditioned medium or to a known concentration of recombinant VEGF. The complex was detected by the biotinylated anti-VEGF antibody, which binds to the captured VEGF. The biotinylated VEGF antibody in turn was detected by streptavidin-alkaline phosphatase and color generating solution. The anti-human VEGF antibody cross-reacts with porcine VEGF.

Determination of MCP-1 in Conditioned Medium by ELISA

The concentration of MCP-1 in conditioned medium was assayed by sandwich enzyme immunoassay kit (R &D Systems, Minneapolis, Minn.): a plate pre-coated with anti human MCP-1 antibody was used to bind MCP-1 in the conditioned medium or to a known concentration of recombinant protein. The complex was detected by the biotinylated anti-MCP-1 antibody, which binds to the captured MCP-1. The biotinylated MCP-1 antibody in turn was detected by streptavidin-alkaline phosphatase and color generating solution. The anti-human MCP-1 antibody cross-reacts with porcine MCP-1.

Results

The BM conditioned medium collected at four weeks increased, in a dose-related manner, the proliferation of PAECs (FIG. 1). This was demonstrated by counting the number of cells directly and by measuring tritiated thymidine uptake (p<0.001 for both measurements). The dose-related response demonstrated a descending limb; proliferation decreased with 200 μL conditioned medium compared to 30 μL and 100 μL (P=0.003 for both comparisons). Similar dose-related results were observed in the tritiated thymidine uptake studies (P=0.03 for 30 μL and 100 μL compared to 200 μL, respectively).

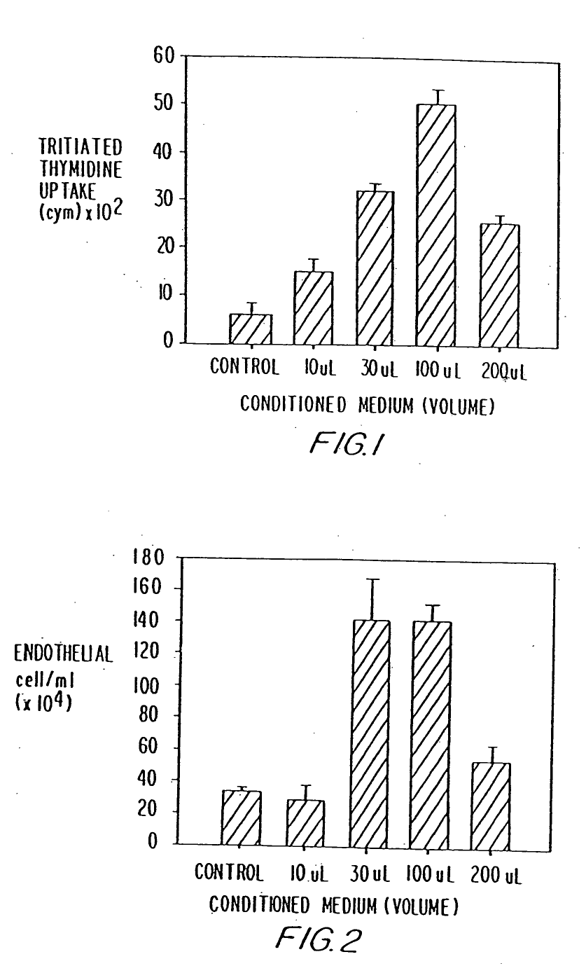

A limited number (5±4%) of freshly aspirated BM cells stained positive for factor VIII. The results are set forth in FIG. 2. This contrasted to 57±14% of the adherent layer of BM cells cultured for 4 weeks, of which 60±23% were endothelial-like cells and 40±28% appeared to be megakaryocytes.

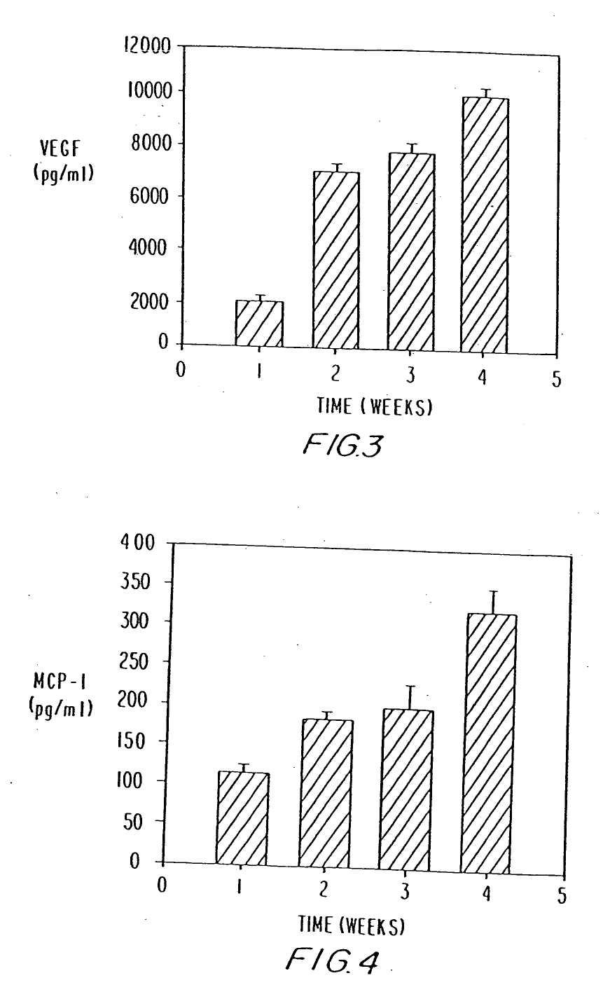

Over a 4-week period, the concentrations of VEGF and MCP-1 in the BM conditioned medium increased gradually to 10 and 3 times the 1st week level, respectively (P<0.00 1 for both comparisons) (FIG. 3). In comparison, VEGF and MCP-1 levels in a control culture medium, not exposed to BM, were 0 and 11±2 pg/ml, respectively, as shown in FIG. 4.

### Example 2

Effects of Hypoxia on VEGF Secretion by Cultured Pig Bone Marrow Cells

It was demonstrated that hypoxia markedly increases the expression of VEGF by cultured bone marrow endothelial cells, results indicating that ex-vivo exposure to hypoxia, by increasing expression of hypoxia-inducible angiogenic factors, can further increase the collateral enhancing effect of bone marrow cells and its conditioned media to be injected in ischemic muscular tissue. Pig bone marrow was harvested and filtered sequentially using 300μ and 200μ stainless steel mesh filters. BMCs were then isolated by Ficoll-Hypaque gradient centrifugation and cultured at 33° C. with 5% CO₂ in T-75 culture flasks. When cells became confluent at about 7 days, they were split 1:3 by trypsinization. After 4 weeks of culture, the BMCs were either exposed to hypoxic conditions (placed in a chamber containing 1% oxygen) for 24 to 120 hrs, or maintained under normal conditions. The resulting conditioned medium was collected and VEGF, MCP-1 were analyzed by ELISA.

Exposure to hypoxia markedly increased VEGF secretion: At 24 hours VEGF concentration increased from 106±13 pg/ml under normoxic, to 1,600±196 pg/ml under hypoxic conditions (p=0.0002); after 120 hours it increased from 4,163±62 to 6,028±167 pg/ml (p<0.001). A separate study was performed on freshly isolated BMCs, and the same trend was found. Hypoxia also slowed the rate of proliferation of BMCs. MCP-1 expression was not increased by hypoxia, a not unexpected finding as its promoter is not known to have HIF binding sites.

### Example 3

Effect of Bone Marrow Cultured Media on Endothelial Cell Tube Formation

It was demonstrated, using pig endothelial cells and vascular smooth muscle cells co-culture technique, that the conditioned medium of bone marrow cells induced the formation of structural vascular tubes in vitro. No such effect on vascular tube formation was observed without exposure to bone marrow conditioned medium. The results suggest that bone marrow cells and their secreted factors exert pro-angiogenic effects.

### Example 4

The Effect of Transendocardial Delivery of Autologous Bone Marrow on Collateral Perfusion and Regional Function in Chronic Myocardial Ischemia Model

Chronic myocardial ischemia was created in 14 pigs by the implantation of ameroid constrictors around the left circumflex coronary artery. Four weeks after implantation, 7 animals underwent transendocardial injections of freshly aspirated ABM into the ischemic zone using a transendocardial injection catheter (2.4 ml per animal injected at 12 sites) and 7 control animals were injected with heparinized saline. At baseline and 4 weeks later, animals underwent rest and pacing echocardiogram to assess regional contractility (% myocardial thickening), and microsphere study to assess collateral-dependent perfusion at rest and during adenosine infusion. Four weeks after injection of ABM collateral flow (expressed as the ratio of ischemic/normal zone×100) improved in ABM-treated pigs but not in controls (ABM: 95±13 vs. 81±11 at rest, P-0.017; 85±19 vs. 72±10 during adenosine, P=0.046; Controls: 86±14 vs. 86±14 at rest, P═NS; 73±17 vs. 72±14 during adenosine, P=0.63). Similarly, contractility increased in ABM-treated pigs but not in controls (ABM: 83±21 vs. 60±32 at rest, P=0.04; 91±44 vs. 35±43 during pacing, P=0.056; Controls: 69±48 vs. 64±46 at rest, P=0.74; 65±56 vs. 37±56 during pacing, P=0.23).

The results indicate that catheter-based transendocardial injection of ABM can augment collateral perfusion and myocardial function in ischemic myocardium, findings suggesting that this approach may constitute a novel therapeutic strategy for achieving optimal therapeutic angiogenesis.

Fourteen specific-pathogen-free domestic pigs weighing approximately 70 kg were anesthetized, intubated, and received supplemental O₂ at 2 L/min as well as 1-2% isoflurene inhalation throughout the procedure. Arterial access was obtained via right femoral artery isolation and insertion of an 8 French sheath. The left circumflex artery was isolated through a left lateral thoracotomy and a metal encased ameroid constrictor was implanted at the very proximal part of the artery. Four weeks after the ameroid constrictor implantation all pigs underwent (1) a selective left and right coronary angiography for verification of ameroid occlusion and assessment of collateral flow; (2) transthoracic echocardiography studies; and (3) regional myocardial blood flow assessment.

Bone Marrow Aspiration and Preparation and Intramyocardial Injection

Immediately after completion of the baseline assessment, all animals underwent BM aspiration from the left femoral shaft using standard techniques. BM was from aspirated 2 sites (3 ml per site) using preservative free heparinized glass syringes (20 unit heparin/1 ml fresh BM). The aspirated bone marrow was immediately macro-filtered using 300μ and 200μ stainless steel filters, sequentially. Then, the bone marrow was injected using a transendocardial injection catheter into the myocardium in 12 sites (0.2 ml per-injection site for total of 2.4 ml) directed to the ischemic myocardial territory and its borderline region.

Echocardiography Study

Transthoracic echocardiography images of short and long axis views at the mid-papillary muscle level were recorded in animals at baseline and during pacing, at baseline and during follow-up evaluation at four weeks after ABM implantation. Fractional shortening measurements were obtained by measuring the % wall thickening (end-systolic thickness minus end-diastolic thickness/end-diastolic thickness)×100. Those measurements were taken from the ischemic territory (lateral area) and remote territory (anterior-septal area). Subsequently, a temporary pacemaker electrode was inserted via a right femoral venous sheath and positioned in the right atrium. Animals were paced at 180/minute for 2 minutes and echocardiographic images were simultaneously recorded.

Regional Myocardial Blood Flow

Regional myocardial blood flow measurements were performed at rest and during maximal coronary vasodilation by use of multiple fluorescent colored microspheres (Interactive Medical Technologies, West Los Angeles, Calif.) and quantified by the reference sample technique (Heymann M A, et al., Prog Cardiovasc Dis 1977; 20:55-79). Fluorescent microspheres (0.8 ml, 5×106 microspheres/ml, 15 μm diameter in a saline suspension with 0.01% Tween 80) were injected into the left atrium via a 6F Judkins left 3.5 diagnostic catheter. Maximal coronary vasodilation was induced by infusing adenosine at a constant rate of 140 μg/kg/min (Fujisawa USA, Deerfield, Ill.) into the left femoral vein over a period of 6 minutes. During the last 2 minutes of the infusion, microsphere injection and blood reference withdrawal were undertaken in identical fashion to the rest study.

Following completion of the perfusion assessment, animals were sacrificed with an overdose of sodium pentobarbital and KCL. Hearts were harvested, flushed with Ringer Lactate, perfusion-fixed for 10-15 minutes, and subsequently immersion-fixed with 10% buffered formaldehyde for 3 days. After fixation was completed, the hearts were cut along the short axis into 7-mm thick slices. The 2 central slices were each divided into 8 similar sized wedges, which were further cut into endocardial and epicardial subsegments. The average of 8 lateral ischemic zone and 8 septal normal zone sub-segments measurements were used for assessment of endocardial and epicardial regional myocardial blood flow. The relative collateral flow was also computed as the ratio of the ischemic zone/non ischemic zone (IZ(NIZ) blood flow.

Histopathology

To assess whether injecting BM aspirate via the use of an injection catheter was associated with mechanical cell damage, standard BM smears were prepared before and after propelling the freshly filtered ABM aspirate through the needle using similar injecting pressure as in the in-vivo study. An independent experienced technician who was blinded to the study protocol performed morphological assessment.

Histopathology assessment was performed on sampled heart tissue. In the pilot study, 7-mm thick short-axis slices were examined under UV light to identify fluorescent-tagged areas. Each identified area was cut into 3 full thickness adjacent blocks (central, right and left) that were immersion-fixed in 10% buffered formaldehyde. Subsequently, each such block was cut into 3 levels, of which 2 were stained with Hematoxylin and Eosin (H&E) and one with PAS. In addition, one fresh fluorescent-labeled tissue block was obtained from the ischemic region of each animal and was embedded in OCT compound (Sakura Finetek USA Inc., Torrance, Calif.) and frozen in liquid nitrogen. Frozen sections of these snap-frozen myocardial tissues were air dried and fixed with acetone. Immunoperoxidase stain was performed with the automated Dako immunno Stainer (Dako, Carpenteria, Calif.). The intrinsic peroxidase and non-specific uptake were blocked with 0.3% hydrogen peroxidase and 10% ovo-albumin. Monoclonal mouse antibody against CD-34 (Becton Dickinson, San Jose, Calif.) was used as the primary antibody. The linking antibody was a biotinylated goat anti-mouse IgG antibody and the tertiary antibody was strepavidin conjugated with horse reddish peroxidase. Diaminobenzidine (DAB) was used as the chromogen and the sections were counterstained with 1% methylgreen. After dehydration and clearing, the slides were mounted and examined with a Nikon Labphot microscope.

In the efficacy study, full-thickness, 1.5 square centimeter sections from the ischemic and non-ischemic regions were processed for paraffin sections. Each of the samples was stained with H&E, Masson's trichrome, and factor VIII related antigen. The immunoperoxidase stained slides were studied for density of endothelial cell population and vascularization. The latter was distinguished from the former by the presence of a lumen. Vascularity was assessed using 5 photomicrographs samples of the factor VIII stained slides taken from the inner half of the ischemic and non-ischemic myocardium. Density of endothelial cells was assessed using digitized images of the same photomicrographs. The density of the endothelial population was determined by Sigma-Scan Pro morphometry software using the intensity threshold method. The total endothelial area for each sample as well as for each specimen were obtained along with the relative percent endothelial area (endothelial area /area of the myocardium studied). The total endothelial area was also calculated as the relative percent of the non-infarcted (viable) area of the myocardium studied. The trichrome stained sections were digitized and the area occupied by the blue staining collagen as well as the total area of the section excluding the area occupied by the epicardium (which normally contained collagen) were measured using Sigma-Scan Pro. The infarcted area was then calculated as the area occupied by the blue staining.

Procedural Data

Intra-myocardial injections either with ABM or placebo were not associated with any acute change in mean blood pressure, heart rate or induction of arrhythmia. All hemodynamic parameters were comparable between the two groups. Pair-wise comparison showed similar hemodynamic parameters within each group in the index compared to the follow-up procedure except for higher initial mean arterial blood pressure at follow-up in the control group (P=0.03) with no subsequent differences during pacing or adenosine infusion.

Myocardial Function

Regional myocardial function assessment is shown in Table 2 below. Preintervention relative fractional wall thickening, expressed as ischemic zone to non-ischemic zone (IZ/IZ) ratio×100, at rest and during pacing, was similar between groups (P=0.86 and 0.96, respectively). At-4 weeks following the intra-myocardial injection of ABM, improved regional wall thickening occurred at rest and during pacing, which was due to an ˜50% increase in wall thickening of the collateral-dependent ischemic lateral wall. No significant changes were observed in the control animals, although a trend towards improvement in wall thickening was noted in the ischemic area during pacing at follow-up.

TABLE 2
Regional Contractility of the Ischemic Wall

| | Baseline | Follow-up | P |
|---|---|---|---|
| Rest | | | |
| ABM (%) | 60 ± 32 | 83 ± 21 | 0.04 |
| Control (%) | 64 ± 46 | 69 ± 48 | 0.74 |
| Pacing | | | |
| ABM (%) | 36 ± 43 | 91 ± 44 | 0.056 |
| Control (%) | 37 ± 56 | 65 ± 56 | 0.23 |

ABM indicates autologous bone marrow.

Myocardial Perfusion Data

Regional myocardial perfusion assessment is shown in Table 3 below. There were no differences between the treated and control groups in the pre-intervention relative transmural myocardial perfusion, IZ/NIZ, at rest and during adenosine infusion (P=O.42 and 0.96, respectively). At 4 weeks following ABM injection, relative regional transmural myocardial perfusion at rest and during pacing improved significantly. This was due to an absolute improvement in myocardial perfusion in the ischemic zone both at rest (an increase of 57%, P=0.08) and during adenosine infusion (37%, P=0.09), while no significant changes were noted in absolute flow to the non-ischemic zone either at rest (increase of 35%, P=0.18) or during adenosine infusion (increase of 25%, P=0.26). The increase in regional myocardial blood flow found in the ischemic zones consisted of both endocardial (73%) and epicardial (62%) regional improvement at rest, with somewhat lesser improvement during adenosine infusion (40% in both zones). At 4 weeks, the control group showed no differences in transmural, endocardial or epicardial perfusion in the ischemic and non-ischemic zones compared to pre-intervention values.

TABLE 3
Regional Myocardial Perfusion

| | Baseline | Follow-up | P |
|---|---|---|---|
| Rest | | | |
| ABM (%) | 83 ± 12 | 98 ± 14 | 0.001 |
| Control (%) | 89 ± 9 | 92 ± 0.1 | 0.43 |
| Adenosine | | | |
| ABM (%) | 78 ± 12 | 89 ± 18 | 0.025 |
| Control (%) | 77 ± 5 | 78 ± 11 | 0.75 |

ABM indicates autologous bone marrow.

Histopathology and Vascularity Assessment

Assessment of BM smears before and after passing the filtrated aspirate through the injecting catheter revealed normal structure, absence of macro-aggregates and no evidence of cell fragments or distorted cell shapes. Histopathology at day I following injections revealed acute lesions characterized by fibrin and inflammatory tract with dispersed cellular infiltration. The infiltrate was characterized by mononuclear cells that morphologically could not be differentiated from a BM infiltrate. Cellularity was maximal at 3 and 7 days and declined subsequently over time. At 3 weeks, more fibrosis was seen in the 0.5 ml injection-sites compared to the 0.2 ml. CD-34 immunostatining, designed to identify BM-derived progenitor cells, was performed in sections demonstrating the maximal cellular infiltrate. Overall, it was estimated that 4-6% of the cellular infiltrate showed positive immunoreactivity to CD-34.

Small areas of patchy necrosis occupying overall <10% of the examined ischemic myocardium characterized the ischemic territory in both groups. The non-ischemic area revealed normal myocardial structure. Changes in the histomorphometric characteristics of the two groups were compared. There were no differences in the total area occupied by any blood vessel as well as the number of blood vessels >50 pm in diameter. However, comparison of the total areas stained positive for factor VIII (endothelial cells with and without lumen) in the ischemic versus the non-ischemic territories revealed differences between the 2 groups. In the ABM group, the total endothelial cell area in the ischemic collateral-dependent zone was 100% higher than that observed in the non-ischemic territory (11.6±5.0 vs. 5.7±2.3% area, P=0.016), whereas there was no significant difference in the control group (12.3±5.5 vs. 8.2±3.1% area, P=0.11). However, ot parameters of vascularity, including % area occupied by any blood vessel and number of blood vessels >50 μm were similar in the ischemic and non-ischemic territories in both groups.

### Example 5

The Effect of Autologous Bone Marrow Stimulated in vivo by Pre-Administration of GM-CSF in Animal Model of Myocardial Ischemia

Chronic myocardial ischemia was created in 16 pigs by the implantation of ameroid constrictors around the left circumflex coronary artery. At four weeks minus 3 days after ameroid implantation, 8 animals underwent subcutaneous injection of GM-CSF for 3 consecutive days (dose 10 μg per day) followed (on the fourth day and exactly 4 weeks after ameroid implantation) by transendocardial injections of freshly aspirated ABM into the ischemic zone using a transendocardial injection catheter (2.4 ml per animal injected at 12 sites) and 8 control animals without GM-CSF stimulation were injected with heparinized saline. At baseline and 4 weeks later, animals underwent rest and pacing echocardiogram to assess regional contractility (% myocardial thickening), and microsphere study to assess collateral-dependent perfusion at rest and during adenosine infusion. Four weeks after injection of ABM collateral flow (expressed as the ratio of ischemic/normal zone×100) improved in ABM-treated pigs but not in controls (ABM: 85-±11 vs. 72±16 at rest, P=0.026; 83±18 vs. 64±19 during adenosine, P=0.06; Controls: 93±10 vs. 89±9 at rest, P=0.31; 73±17 vs. 75±8 during adenosine, P=0.74). Similarly, contractility increased in ABM-treated pigs but not in controls (ABM: 93±33 vs. 63±27 at rest, P=0.009; 84±36 vs. 51±20 during pacing, P=0.014, Controls: 72±45 vs. 66±43 at rest, P=0.65; 70±36 vs. 43±55 during pacing, P=0.18).

The results indicate that catheter-based transendocardial injection of ABM prestimulated in vivo by GM-CSF administered systemically for 3 days, can augment collateral perfusion and myocardial function in ischemic myocardium, findings suggesting that this approach may constitute a novel therapeutic strategy for achieving optimal therapeutic angiogenesis.

### Example 6

Pig Bone Marrow Culture

Bone marrow cells (BMCs) are harvested under sterile conditions from pigs in preservative free heparin (20 units/ml BM cells) and filtered sequentially using 300μ and 200μ stainless steel mesh filters. BMCs are then isolated by Ficoll-Hypaque gradient centrifugation, seeded in T-75 flasks, and cultured overnight in long-term culture medium (LTCM) (Stem Cell Tech, Vancouver, British Columbia, Canada) at 33° C. with 5% CO₂ in T-75 culture flasks. The medium is then changed and the non-attaching cells washed out. The attached cells (i.e., “early attaching cells”) are mostly monocytes, endothelial precursor cells, or other hemopoietic lineage cells. Among the monocytes in early attaching cells are marrow-derived stromal cells. By lac-Z staining testing, these cells have been shown to be permissive for adenovirus by expression of the marker protein.

The seeding density of the BMCs in each culture dish is 7×10⁶/ml. When the cells become confluent at about 7 days, they are split 1 to 3 by 0.25% trypsin. Passages 3-8 were used for this study.

Adenovirus Transfection

BMCs are first cultured in 6-cm Petri dishes for 3 to 14 days to allow for production of a lining of early attaching cells that adhere to the Petri dish. The non-adherent cells are washed away the day after initial seeding. Then the early attaching cells are inoculated with a vector encoding one or more cytokines, growth factors, or other mammalian angiogenesis promoting factors, such as, but not limited to, the transcription factors HIP-I or HIF-2. This inoculation can occur from 3 to 28 days after seeding, for example 3 to 12 days or 3 to 8 days. The virus is washed out from the transfected cells about 2 hours to 3 days after inoculation. The transfected cells can then be injected into the patient's target tissue, such as the muscle of heart or leg.

### Example 7

MSCs Have the Capacity to Secrete Biologically Active Collateral-Enhancing Factors in vitro.

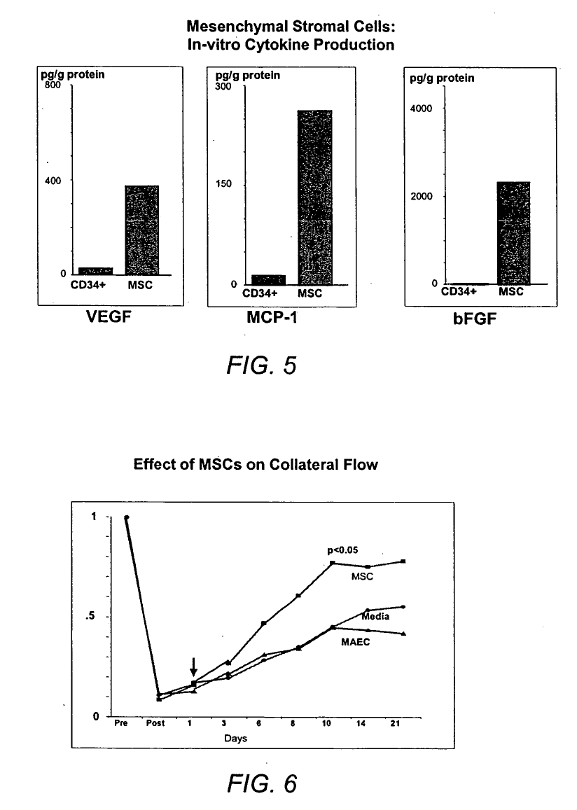

As a first test of the feasibility of the hypotheses that 1) conditioned medium derived from bone marrow-derived cells can, by itself, enhance collateral flow and 2) HIF-1 transduction of MSCs increases the angiogenic potential of the cells, murine MSCs were isolated and the conditioned medium was serially analyzed for cytokine production (FIG. 5).

More particularly, mononuclear marrow cells were harvested from the femur and tibiae of mice and the mononuclear fraction separated using a Ficoll density gradient. The cells were cultured for 10 days and the CD34 minus/CD45 minus cells were isolated from the heterogeneous cultured cells using a double magnetic bead technique. This isolation procedure involves negatively selecting cells not expressing cell markers CD34 and CD45 by using magnetic beads labeled with commercially available antibodies to these markers.

MSCs were purified from the heterogeneous cultured cells. The CD34 minus-/CD45 minus-fraction was isolated by labeling with FITC-labeled anti-CD34 antibody (Pharmingen, San Diego, Calif.) followed by simultaneous incubation with anti-FITC and anti-CD45 magnetic beads (Miltenyi Biotech, Sunnyvale, Calif.). Cells were passed through a magnetic column and the double-negative fraction collected. Subsequently, the bead-negative and bead-positive populations were separately cultured. The bead-negative population demonstrated typical fibroblastic morphology of the MSCs, while the bead-positive population appeared to mainly consist of small, spherical cells consistent with lymphohematopoietic cells (FIGS. 5A and 5B). FACS analysis was performed and demonstrated that cells did not express the surface makers CD31, CD34, CD45, and CD117 typical of lymphohematopoietic cells, but did express high levels of CD44 (95±0.6%), CD90 (99.1±0.1%), and CD105 (89±2.1%) typical of marrow derived-stromal cells.

(These CD34 minus/CD45 minus cells are also referred to herein as “marrow-derived stromal cells”, or “MSCs”). The isolated MSCs were replated, and the conditioned media subsequently collected for 24 hours.

Conditioned media prepared as above was analyzed for the presence of angiogenic cytokines by ELISA. Cytokine levels were corrected for total cell culture protein. The data reflect at least 3 different cell populations, with each population containing cells pooled from 2 mice. The results show (FIG. 5) that MSCs express such known collateral-enhancing factors as VEGF, MCP-1, and bFGF (also, angiopoietin-1 and PDGF (not shown)). In contrast, CD34+ cells (progenitor endothelial cells) do not express these factors.

The functional capacity of the cytokines secreted into the medium of cultured MSCs was also tested by testing their capacity to cause endothelial cell proliferation. MSC-conditioned media prepared as above was collected and found to indeed increase the proliferation of cultured human umbilical vein endothelial cells. MAECs or SMC's (1×10⁴/well) were plated in 24-well plates in MEM with 0.1% fetal calf serum for 24-hours. The media was then replaced with varying dilutions of MSC^{CM} or control wells of DM-10 only. Cultures were continued for 72-hours, after which the cells were recovered and counted using a Coulter counter. Data is reported as the mean % change in proliferation when compared with control.

MSCs and Conditioned Medium from MSCs Increase Collateral Flow in the Mouse Ischemic Hindlimb.

Twelve week-old Balb/C male mice underwent right distal femoral artery ligation using a method known in the art. Twenty-four hours later, mice were randomized to 3 groups—one group received 1×10⁶ MSCs prepared as above described from syngeneic mice, one group received 1×10⁶ mature endothelial cells isolated from syngeneic mice, and one group received non-conditioned media injected into the adductor muscles of the ischemic hindlimb. Laser Doppler perfusion imaging (LDPI) was utilized to follow ischemic hindlimb flow recovery over the ensuing 28 days (FIG. 6).

The results of these tests shown in FIG. 6 demonstrate that injection of MSCs into the adductor muscles of the ischemic hindlimb significantly increased collateral flow, an effect not seen by injecting mature endothelial cells. Most importantly, it was found that when the conditioned media itself (devoid of cells) was injected into the ischemic hindlimb, collateral flow increased. This was the first proof-of-concept that it is possible to augment collateral development by injecting conditioned medium of bone marrow-derived cells into the region of developing collaterals.

Confirmation of Cellular Survival and Gene Product Expression Following Transduction of MSCs.

As an initial step to determine whether MSCs provide an appropriate target for genetic alteration, the viability of MSCs in-situ following ex-vivo transduction with an adenoviral vector was examined. To this purpose, two separate experiments were performed, one utilizing an adenovirus comprising a gene encoding for Green Fluorescent Protein (GFP) and one comprising a gene encoding β-galactosidase. MSCs prepared as above were transduced ex-vivo Preliminary studies determined that over 90% of MSCs were successfully transduced with an adenovirus containing a reporter transgene at an MOI of 150 (data not shown). To track protein expression, cells were incubated with Ad.GFP or Ad.β-galactosidase at an MOI of 150 for 2-hours, rinsed three times, recovered and immediately injected into the adductor muscle (24-hours post-surgery). To follow the fate of injected GFP+/MSCs, multiple sections of adductor and calf muscle were examined using a Nikon inverted fluorescent microscope. To follow the fate of β-gal+/MSCs, sections were developed with a commercially available X-gal kit (Invitrogen) and immediately injected into the adductor muscle of mice that had undergone femoral artery ligation 24-hours previously. Mice were sacrificed at day-3, day-7 and day-14. Adductor muscle sections were subsequently either examined under a fluorescent microscope or stained with X-gal depending on the appropriate protocol as known in the art.

At day-3, few cells were found that expressed the gene-of-interest. However, by day-7 and maintained through to day-14, many cells expressing the gene-of-interest were found distributed throughout the adductor tissue.

Therefore, this experiment not only confirmed cell viability and preservation of the transcriptional/translational mechanism, but also demonstrated that MSCs can be used as a vector to introduce genes-of-interest into a particular tissue, such as muscle tissue.

HIF-1α/VP16 Transfection of MSCs in vitro Leads to an Increase in Collateral-Enhancinig-Related Factors Greater than Those Induced by Hypoxia.

Murine MSCs were isolated and plated as described above. Three groups of MSCs were compared. Group 1—MSCs cultured under normoxic conditions; Group 2—MSCs cultured in 1% O₂; Group 3—MSCs transfected with an adenovirus encoding HIF-1α/VP16 prepared as described above. MSCs were incubated with the virus at a multiplicity of infection of 200 for 2 hr, followed by 48 hr of culture to allow time for gene expression.

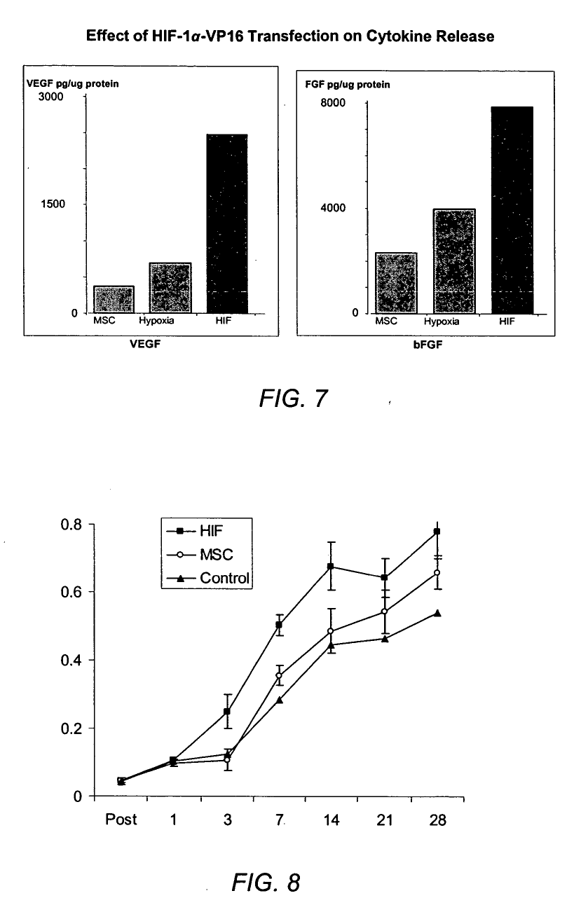

The culture-conditioned media was subsequently collected for 24 hr from all 3 groups of cells. Using commercially available ELISA kits, media was analyzed for the presence of angiogenic cytokines VEGF and ∂-FGF. Cytokine levels were corrected for total cell culture protein. The results shown in FIG. 7 demonstrate that HIF-1I/VP16 transfection increases expression and secretion by MSCs of both VEGF and ∂FGF to levels substantially greater than those achieved by hypoxia.

Medium bathing these cultured cells (MSC conditioned medium, or MSC^{CM}) was also added to cultures of endothelial cells (EC) and smooth muscle cells (SMC) to assess the effect of MSC^{CM} on cell proliferation. Mouse aortic endothelial cells (MAECs) were isolated as follows. Under sterile conditions, murine thoracic aortas were dissected (n=10), the adventitia removed, and then cut into 1-2 mm rings. Rings were then incubated with 0.25% trypsin for 20 minutes at 37° C., followed by washing and harvesting of floating cells. These were cultured in Minimal Essential Media supplemented with 10% FBS. Cells were uniformly positive for Factor VIII. Smooth muscle cells (SMC's) were isolated using a modification of a previously described protocol.⁸ Briefly, after collecting MAECs as above, collagenase in Hanks Balanced Salt Solution (1 mg/ml) was added and incubated in 37° C. for up to 3 hours with gentle agitation every 15-30 min. Floating cells were again harvested, washed and re-suspended in Medium 199 supplemented with 10% FBS. Cells stained uniformly for smooth-muscle actin. Passages 3-8 for both cells were used for the purposes of the study.

When compared to MSC^{CM} from MSCs under conditions of normoxia or hypoxia, MSC^{CM} from HIF-1α/VP16-transduced MSCs increased EC proliferation (290% vs. 31% vs. 79% compared to proliferation in control media, p<0.001) and SMC proliferation (220% vs. 26% vs. 58%, p<0.001).

HIF-1α/VP16 Transfection of MSCs Leads to an Increase in Collateral Flow.

The effects of HIF-1I/VP16 transduction of MSCs on collateral flow in a mouse model of hindlimb ischemia was studied next. One group of animals (as above) received 1×10⁵ non-transduced MSCs, one group received HIF-1I/VP16-transduced cells and a third group received media. Flow in the ischemic limb was monitored as described above. The results collected over the course of 21 days (FIG. 8) showed that mice treated with transduced MSCs demonstrated a consistently greater increase in collateral flow recovery than that observed in mice treated with non-transduced MSC.

In summary, these experiments show that 1) conditioned media itself (devoid of cells), when injected into the ischemic hindlimb, enhances collateral flow (the first proof-of-concept that it is possible to augment collateral development by injecting conditioned medium of bone marrow-derived cells into the region of developing collaterals); 2) transfection with HIF-1α/VP16 in an adenoviral vector significantly and markedly enhances the in-vitro angiogenic effects of MSCs. Importantly, in vivo studies indicate that this strategy results in an increase in the collateral-improving effects over that achieved by injection of MSCs alone. These studies indicate that transduction of MSCs with HIF-1α (and most probably also with genes encoding other angiogenic-related cytokines, such as the FGF family of proteins, and NOS) will optimize the collateral-enhancing effects of a cell-based strategy for increasing collateral flow in ischemic tissue.

Treatment of a Human Patient

Bone marrow (˜5 ml) will be aspirated from the iliac crest using preservative-free heparinized glass syringes (20 unit heparin/1 ml fresh BM). The aspirated bone marrow will be immediately macro-filtered using 300μ and 200μ stainless steel filters, sequentially. The bone marrow will be kept in standard anticoagulation/anti-aggregation solution (containing sodium citrate and EDTA) and kept in 4° C. in sterile medium until the time of its use. An experienced hematologist will perform the procedure under sterile conditions. The bone marrow smear will be evaluated to confirm a normal histomorphology of the bone marrow preparation.

Autologous or allogenic cell-free conditioned medium will be prepared as described above.

Any of several procedures for delivery of an agent to the myocardium can be used for delivery of the cell-free conditioned medium to the patient. These include direct transepicardial delivery, as could be achieved by a surgical approach (for example, but not limited to, a transthoracic incision or transthoracic insertion of a needle or other delivery device, or via thoracoscopy), or by any of several percutaneous procedures. Following is one example of percutaneous delivery. It should be emphasized that the following example is not meant to limit the options of delivery to the specific catheter-based platform system described in the example—any catheter-based platform system can be used.

Using standard procedures for percutaneous coronary angioplasty, an introducer sheath of at least SF is inserted in the right or left femoral artery. Following insertion of the arterial sheath, heparin is administered and supplemented as needed to maintain an ACT for 200-250 seconds throughout the LV mapping and injection of cell-free conditioned medium. ACT will be checked during the procedure at intervals of no longer than 30 minutes, as well as at the end of the procedure to verify conformity with this requirement.

Left ventriculography is performed in standard RAO and/or LAO views to assist with guidance of NOGA-STAR3 and injection catheters, and an LV electromechanical map is obtained using the NOGA-STAR3 catheter. The 8F INJECTION-STAR catheter is placed in a retrograde fashion via the femoral sheath to the aortic valve. After full tip deflection, the rounded distal tip is gently prolapsed across the aortic valve and straightened appropriately once within the LV cavity.

The catheter (incorporating an electromagnetic tip sensor) is oriented to one of the treatment zones (e.g., anterior, lateral, inferior-posterior or other). Utilizing the safety features of the NOGA3 system, needle insertion and injection is allowed only when stability signals will demonstrate an LS value of <3. A single injection of 0.2 cc of cell-free conditioned medium will be delivered via trans-endocardial approach to the confines of up to two treatment zones with no closer than 5 mm between each injection site. The density of injection sites will depend upon the individual subject's LV endomyocardial anatomy and the ability to achieve a stable position on the endocardial surface without catheter displacement or premature ventricular contractions (PVCs).

According to the invention methods, in one embodiment effective amounts of autologous or allogenic cell-free conditioned medium obtained from angiogenic progenitor cells is administered for treatment. As would be appreciated by experienced practitioners, the amount administered will depend upon many factors, including, but not limited to, the intended treatment, the severity of a condition being treated, the size and extent of an area to be treated, etc. With regard to treatment according to the invention, a representative protocol would be to administer quantities of from about 0.2 to about 0.5 ml of cell-free conditioned medium in each of from about 12 to about 25 injections, for a total of from about 2.4 to about 6 ml of cell-free conditioned medium being administered. Each dose administered could preferably comprise from about 1 to about 2 percent by volume of heparin or another blood anticoagulant, such as coumadin. When the cell-free conditioned medium has been stimulated and/or is being administered in combination with other angiogenic factors as described herein, the quantity of cell-free conditioned medium used should be approximately the same in each dose and/or the total of the cell-free conditioned medium administered should be about the same as described above. The concentration of the cell-free conditioned medium can be adjusted by the practitioner by concentration to contain an effective amount of the cell-free conditioned medium depending upon the number and spacing of the injection sites as well as the requirements of the particular condition being treated, as described above.

The cell-free conditioned medium, with or without a stimulatory agent in any of its delivery forms, or with or without the allogenic or autologous progenitor cells used to prepare the conditioned medium having been transfected with a vector carrying a transgene that is designed to enhance the angiogenesis effect of the cell-free conditioned medium, will be injected into the heart muscle, i.e., in therapeutic myocardial angiogenesis or therapeutic myogenesis, using either any catheter-based trans-endocardial injection device or via a surgical (open chest) trans-epicardial thoracotomy approach, or any other approach that allows for transepicardial delivery. In the case of treatment of limb ischemia the cell-free conditioned medium will be transferred by a direct injection of the cell-free conditioned medium or it elements, with or without ex-vivo or in vivo stimulation in any of its delivery forms, into the muscles of the leg.

The volume of injection per treatment site will probably range between 0.1-5.0 cc per injection site, dependent upon the specific cell-free conditioned medium product and severity of the ischemic condition and the site of injection. The total number of injections will probably range between 1-50 injection sites per treatment session.

In another embodiment, an equivalent dosage of the cell-free conditioned medium (taking into account dilution thereof by the patient's blood), with or without a stimulatory agent in any of its delivery forms, or with or without the allogenic or autologous progenitor cells used to prepare the conditioned medium having been transfected with a vector carrying a transgene that is designed to enhance the angiogenesis effect of the cell-free conditioned medium, will be injected into the vasculature adjacent to the treatment site. In this case blood flow will deliver the cell-free conditioned medium to the treatment site.

The present invention may be embodied in other specific forms without departing from the spirit or central attributes thereof. Thus, the foregoing description of the present invention discloses only exemplary embodiments thereof, and other variations are contemplated as being within the scope of the present invention. Accordingly, the present invention is not limited to the particular embodiments that have been described in detail herein. Rather, reference should be made to the appended claims as indicative of the scope and content of the invention.

The preceding specific embodiments are illustrative of the practice of the invention. It is to be understood, however, that other expedients known to those skilled in the art or disclosed herein, may be employed without departing from the spirit of the invention or the scope of the appended claims.

What is claimed is:

1. A method for producing a composition useful for enhancing development of collateral blood vessels in a patient in need, said method comprising:
a) growing isolated autologous or allogenic progenitor cells selected from bone marrow, adipose, or peripheral blood progenitor cells under suitable culture conditions in a suitable growth medium for a period of time sufficient to promote secretion by the progenitor cells of mixed secretion products, thereby obtaining conditioned medium; and
b) processing the conditioned medium to obtain substantially cell-free conditioned medium.

2. The method of claim 1, wherein the progenitor cells are grown in culture for about 7 to 10 days.

3. The method of claim 1, wherein the progenitor cells are grown in culture for about 7 to 25 days

4. The method of claim 3, further comprising exposing the progenitor cells to hypoxic atmosphere.

5. The method of claim 4, wherein the exposing is for about 24 to about 72 hours.

6. The method of claim 4, wherein the hypoxic atmosphere comprises about 1% to about 3% oxygen.

7. The method of claim 1, wherein the progenitor cells comprise a mixture of progenitor cell types.

8. The method of claim 1, wherein the progenitor cells comprise CD34⁺ or CD34⁻ progenitor cells.

9. The method of claim 1, further comprising prior to a),
obtaining allogenic bone marrow from a human donor; and
culturing the allogenic bone marrow under suitable culture conditions for a period of time sufficient to promote production of the progenitor cells by cells in the bone marrow.

10. The method of claim 9, wherein the culturing of the bone marrow cells is for 7 to 25 days.

11. The method of claim 9, wherein the bone marrow is filtered to remove unwanted particles larger than from about 300μ to about 200μ prior to the culturing.

12. The method of claim 1, wherein the method further comprises subjecting the progenitor cells to an hypoxic atmosphere during the growing.

13. The method of claim 1, further comprising subjecting the progenitor cells to hypoxic atmosphere or to contact with a hypoxia inducing factor-1 (HIF-1) or Monocyte Chemoattractant Protein 1 (MCP-1).

14. The method of claim 13, wherein the HIF-1 is an HIF1α/VP16 construct that is stable under non-hypoxic conditions.

15. The method of claim 1, further comprising transfecting at least some of the progenitor cells with one or more polynucleotide encoding an angiogenic protein selected from an HIF-1, EPAS1, Monocyte Chemoattractant Protein 1 (MCP-1), granulocyte-monocyte colony stimulatory factor (GM-CSF), PR39, a fibroblast growth factor (FGF), or a nitric oxide synthase (NOS) and culturing the transfected cells to produce the angiogenic protein in culture prior to b).

16. The method of claim 1, further comprising subjecting the cell-free conditioned medium to stimulation with hypoxic atmosphere or at least one angiogenic protein selected from EPAS 1, MCP-1, GM-CSF, PR39, a FGF or a NOS.

17. The method of claim 1, further comprising prior to a):
obtaining allogenic adipose tissue from a donor; and
processing the adipose tissue to obtain the progenitor cells therefrom prior to culturing the progenitor cells.

18. The method of claim 1, wherein the processing comprises filtering the medium using a filter sized to remove cells therefrom.

19. A therapeutic composition useful for enhancing development of collateral blood vessels in a patient in need when injected into to a site of impaired blood flow, said composition comprising:
a cell-free conditioned medium comprising an effective amount of mixed secretion products of autologous or allogenic angiogenic progenitor cells.

20. The composition of claim 19, wherein the angiogenic progenitor cells are obtained from bone marrow cells, peripheral blood cells or adipose cells.

21. The composition of claim 19, wherein the angiogenic progenitor cells comprise CD34⁺/CD34⁻ cells.

22. The composition of claim 19, wherein the progenitor cells are allogenic to the patient.

23. The composition of claim 22, wherein the composition is lyophilized.

24. The composition of claim 22, wherein the composition is frozen.

25. The composition of claim 19, wherein the progenitor cells are autologous to the patient.

26. The composition of claim 19, wherein the progenitor cells are obtained from adipose tissue or blood from the donor.

27. The composition of claim 19, wherein the progenitor cells are obtained from bone marrow aspirated from the donor.

28. The composition of claim 19, further comprising one or more angiogenic proteins selected from an HIF-1 or MCP-1.

29. The composition of claim 19, further comprising one or more angiogenic proteins selected from EPAS1, MCP-1, GM-CSF, PR39, a FGF or a NOS.

30. The composition of claim 19, wherein the donor is a human.

31. The composition of claim 19, further comprising a container containing the cell-free medium.

32. A kit comprising:
the composition of claim 19 contained in a container; and
an instruction for using the composition to enhance collateral blood vessel development at a site of impaired blood flow in a mammal.

33. The kit of claim 32, wherein the progenitor cells are human.

34. The kit of claim 33, wherein the progenitor cells are CD34+ progenitor cells.

35. A method for enhancing collateral blood vessel formation in a patient in need thereof, said method comprising:
directly administering to tissue having impaired blood flow in the patient or tissue adjacent thereto an amount of the composition of claim 19 sufficient to enhance angiogenesis and collateral blood vessel formation in the tissue.

36. The method of claim 35, wherein the composition is administered to two or more sites in the tissue.

37. The method of claim 35, wherein the tissue is myocardial or peripheral limb tissue.

38. The method of claim 36, wherein the administration is by injection directly into the sites.

39. The method of claim 37, wherein the tissue is myocardial and the injection is by catheter.

40. The method of claim 38, wherein the composition is injected directly into heart or leg muscle to promote angiogenesis therein.

41. The method of claim 35, wherein the composition is administered by catheter or needle into the blood stream for delivery to the tissue.

42. The method of claim 35, wherein the patient is elderly and the allogenic progenitor cells are obtained from a young, healthy donor.
